(19) United States
(12) Patent Application Publication
(10) Pub. No.: US 2008/0176305 A1
(43) Pub. Date: Jul. 24, 2008

(54) FUNCTIONAL COMPOSITION

(75) Inventors: Takuya Sato, Noda (JP); Daisuke Kaneko, Noda (JP); Yasutomo Shinohara, Noda (JP); Satoshi Fukuda, Noda (JP); Ryota Kanegae, Noda (JP); Ikuko Masuda, Noda (JP)

Correspondence Address: YOUNG & THOMPSON, 209 Madison Street, Suite 500, ALEXANDRIA, VA, 22314, US

(73) Assignee: Kikkoman Corporation, Noda (JP)

(21) Appl. No.: 11/958,401

(22) Filed: Dec. 18, 2007

(30) Foreign Application Priority Data
Dec. 20, 2006 (JP) 2006-341989
Jan. 24, 2007 (JP) 2007-013905
Jan. 25, 2007 (JP) 2007-015008
Aug. 23, 2007 (JP) 2007-216553

(51) Int. Cl. C12P 1/04

(52) U.S. Cl. 435/170

(57) ABSTRACT

A functional composition containing as an active ingredient a lactic acid fermented product obtained by fermenting a component of soybean origin using a lactic acid bacterium belonging to the genera Lactobacillus, Leuconostoc, or Pediococcus. It has various activities such as growth hormone secretagogue activity, fatigue accumulation preventing activity, circadian rhythm regulatory activity, reproductive behavior inducing activity, and so forth and is useful as a growth hormone secretagogue, a fatigue accumulation preventing composition, a circadian rhythm regulator, or a reproductive behavior-inducing composition.

### FIELD OF THE INVENTION

This invention relates to a functional composition containing a lactic acid fermented product of a component of soybean origin as an active ingredient. The functional composition of the invention shows various activities such as growth hormone secretagogue activity, fatigue accumulation-preventing activity, circadian rhythm regulatory activity, reproductive behavior-inducing activity, and so forth and is useful as a growth hormone secretagogue, a fatigue accumulation preventing composition, a circadian rhythm regulator, or a reproductive behavior-inducing composition.

As used herein, the term “growth hormone secretagogue activity” means the capability of the functional composition to promote the secretion of growth hormone from pituitary gland cells or to increase the blood growth hormone level.

As used herein, the term “fatigue accumulation preventing activity” means the capability of the functional composition to prevent accumulation of physical fatigue and to enhance voluntary motor activity or voluntary motivation.

The term “circadian rhythm regulatory activity” as used herein means the capability of the functional composition to induce changes of circadian rhythm-controlling gene expression.

The term “reproductive behavior inducing activity” as used herein means the capability of the functional composition to enhance the motivation for reproductive behavior or increase the number of reproductive behaviors in male animals.

### BACKGROUND OF THE INVENTION

Growth hormone is an important factor controlling the growth of a living body. Growth hormone also relates to the metabolism of a living body, participating in increasing the rate of protein synthesis, decreasing the rate of hydrocarbon utilization, and increasing the circulation of free fatty acids and utilization of fatty acids. Human growth hormone consists of 191 amino acid residues. It is secreted from the pituitary gland and delivered to every part of the body through the bloodstream. An increased blood level of growth hormone as a result of promoted secretion causes such physiological phenomena as an increase in muscle mass, an increase in skin elasticity, an improvement of cardiopulmonary function, and an increase of bone density. Therefore, an excellent growth hormone secretagogue is useful as a pharmaceutical or a functional food.

Known growth hormone secretagogues include arginine, L-3,4-dihydroxyphenylalanine (L-DOPA), glucagon, vasopressin, and insulin. It is also known that ingestion of soybean 7S globulin brings about an increase of growth hormone level as reported in The Proceedings of the 2003 Annual Meeting of the Agricultural Chemical Society of Japan, p. 57.

Physical exercise stimulates metabolism and improves cardiopulmonary function and is therefore vital to health maintenance. As used herein the term “voluntary motor activity” refers to exercise that is not caused forcibly but from one's own motive.

When a living body is in a fatigued condition, voluntary motor activity reduces. Relieving fatigue results in an increase of voluntary motor activity. Voluntary motor activity is therefore considered to be a measure of fatigue accumulation.

To prevent fatigue accumulation leads to securing an adequate amount of daily exercise and contributes to improvement and maintenance of motor function of a living body. Accordingly, the composition that prevents fatigue accumulation is useful as a pharmaceutical or a functional food.

It has been known that voluntary motor activity is stimulated by the peptides or amino acids formed as a result of protein decomposition with thiol protease and/or serine protease. These peptides and amino acids are known to promote voluntary motor activity through the same fatigue-relieving effect as with such functional foods as Korean ginseng, royal jelly, propolis protein as reported, e.g., in JP 2005-239579 and WO 03/011056. However, the functional foods described above are too expensive to be eaten daily. A less expensive functional food that can be ingested in our daily life has been awaited.

A circadian rhythm is a roughly-24-hour cycle in the physiological processes of living beings. It is regulated by circadian rhythm controlling genes. Examples of known circadian rhythm controlling genes in mammals include Rev-erb genes, Clock gene, Per genes, Bmal 1 genes, and Cry genes. It has been revealed that the diurnal change pattern of expressions of these genes largely differ according to the organ or tissue of a living body. It is thought that to vary the expression of these genes systemically or topically will allow for circadian rhythm regulation as described in Bookout et al., Cell, p. 789, 2006.

Rev-erb α is identified to be one of nuclear receptors and expressed in a wide range of organs including the kidneys and liver (see, Remakrishnan et al., Nuclear Receptor Signaling, 4, 2006). Rev-erb α has been identified as a critical regulator of circadian rhythm. Rev-erb α negatively controls the circadian rhythm controlling genes such as Clock and Bmal 1 genes. This is supported by the fact that Rev-erb α knockout mice show a disrupted circadian rhythm compared with normal mice.

It has been revealed that lithium as a drug for treating bipolar disorder demonstrates its effect via Rev-erb α as reported in Yin et al., Science, 311, p. 1002, 2006. Thus, Rev-erb α is considered to have a circadian rhythm regulatory function and to be a target factor in the treatment of bipolar disorder.

There is a report that a fibrate, a hypolipidemic drug, increases Rev-erb α expression in liver cells. In Rev-erb α gene expression, transcription of Rev-erb α is inhibited by the Rev-erb α itself, which binds to the Rev-erb α gene binding site of the promoter of Rev-erb α gene. However, a fibrate causes PPAR α and RXR α to bind to the Rev-erb α binding site of the promoter. That is, because a factory other than Rev-erb α binds to the Rev-erbα binding site, it is considered that the Rev-erb α transcription is not inhibited, whereby Rev-erb a expression increases (see Gervois et al., Molecular Endocrinol., 13, p. 400, 1999) It is therefore expected that PPAR α and RXR α genes may be used to control the amount of Rev-erb α gene expression and that a blood lipid (triglycerides) lowering effect may be exerted through the same mechanism as of fibrates.

Microarray analysis revealed that liver gene expression between rats fed a soy protein diet and rats fed a casein diet has shown no significant difference in Rev-erb α expression (see Tachibana et al., J. Agric. Food Chem., Vol. 53, p. 4253, 2005).

Disruption of circadian rhythm induces sleep disorder, insomnia, autonomic dystonia, bipolar disorder, and so forth. Causes of disruption of circadian rhythm include an irregular work schedule (e.g., night work) and a long-distance flight.

It is possible to normalize disrupted circadian rhythm by regulating the circadian rhythm controlling genes. Normalizing disrupted circadian rhythm is contributory to the treatment and prevention of sleep disorder, insomnia, bipolar disorder, or a like disease. Therefore, an excellent functional composition is useful as a pharmaceutical or a functional foodstuff.

It is known that circadian rhythm is normalized by arachidonic acid and/or a compound having arachidonic acid as a constituent fatty acid or 5-hydroxytryptophane as described in JP 2006-521369A and JP 2003-81829A. An expression enhancer for Per gene, one of the circadian rhythm controlling genes, is also known as disclosed in JP 2003-335669A.

Reproductive behavior is indispensable for procreation and is recognized, in humans, to be an important behavior for satisfying sexual desire.

A good sex life is thought to contribute to an improved quality of life (QOL) or a happy marriage. However, sex life problems such as sexual dysfunction (including erectile dysfunction (hereinafter ED)) and loss of sexual desire have been reported. Stress, mental fatigue or physical fatigue is deemed to be a great factor causing ED or loss of sexual desire. Lifestyle-related diseases such as diabetes and obesity are also regarded as a cause of sexual dysfunction.

Decline in birth rate is a serious problem in Japan, and to improve ED or loss of sexual desire seems to be one of the solutions addressing this problem. Sildenafil has been developed as a therapeutic agent for ED and prescribed for patients with ED. However, reports have indicated that sildenafil has a temporary side effect and an interaction with other drugs so that extreme caution is required in prescribing. It has therefore been desired to develop a reproductive behavior-inducing composition which is obtained from a food material that has been safely consumed for a long period of time and which is easy to ingest.

Nutmeg is known to induce reproductive behavior in rats (see BMC Complementary and Alternative Medicine Vol. 5, pp. 16-22, 2005). A sexual drive enhancer containing a garlic extract or a scallop (Patinopecten yessoensis) extract is disclosed in JP 10-306032A and JP 2005-289862, respectively.

### DISCLOSURE OF THE INVENTION

The present invention is contemplated to provide a novel functional composition having various activities such as growth hormone secretagogue activity, fatigue accumulation preventing activity, circadian rhythm regulatory activity, reproductive behavior inducing activity, and so forth, highly safe, inexpensive, and easy to ingest. The invention is also contemplated to provide a process of preparing the functional composition.

As a result of extensive investigations, the present inventors have found that a lactic acid fermented product obtained by fermenting a component of soybean origin using a specific lactic acidbacterium exhibits various activities, and completed the present invention. The present invention provides the following:

(1) A functional composition containing as an active ingredient a lactic acid fermented product obtained by fermenting a component of soybean origin using a lactic acid bacterium belonging to the genera Lactobacillus, Leuconostoc, or Pediococcus.
(2) The functional composition of (1) above, wherein the component of soybean origin is soy milk.
(3) The functional composition of (1) or (2) above, wherein the lactic acid bacterium is Leuconostoc pseudomesenteroides ATCC 12291.
(4) The functional composition of anyone of (1) to (3) above, which is a functional food.
(5) The functional composition of any one of (1) to (3) above, which is a growth hormone secretagogue.
(6) The functional composition of any one of (1) to (3) above, which is a fatigue accumulation preventing composition.
(7) The functional composition of anyone of (1) to (3) above, which is a reproductive behavior inducing composition.
(8) The functional composition of any one of (1) to (3) above, which is a composition regulating circadian rhythm through an increase of Rev-erb α and/or PPAR a mRNA expression or a decrease of Clock or Bmal 1 mRNA expression.
(9) The functional composition of (8) above, which is used to prevent or reduce a symptom attributed to disrupted circadian rhythm.
(10) The functional composition of (9) above, wherein the symptom attributed to disrupted circadian rhythm is sleep disorder, insomnia, autonomic dystonia, bipolar disorder, or jet lag.
(11) A process of preparing the functional composition of any one of (1) to (10) above, comprising inoculating a lactic acid bacterium belonging to the genera Lactobacillus, Leuconostoc, or Pediococcus to a component of soybean origin, then subjecting the component of soybean origin to fermentation, and collecting the fermented product.
(12) The process of (11) above, wherein the lactic acid bacterium is Leuconostoc pseudomesenteroides ATCC 12291.

The functional composition of the invention has various activities such as growth hormone secretagogue activity, fatigue accumulation preventing activity, circadian rhythm regulatory activity, reproductive behavior inducing activity, and so forth and is highly safe, inexpensive, and easy to ingest. The functional composition is useful as a growth hormone secretagogue, a fatigue accumulation preventing composition, a circadian rhythm regulator, or a reproductive behavior-inducing composition.

When the functional composition with growth hormone secretagogue activity is used as a growth hormone secretagogue, it exhibits physiological actions such as increase in muscle mass, increase in skin elasticity, improvement of cardiopulmonary function, and increase of bone density through its in vivo growth hormone secretagogue activity thereby to bring about good effects on heath maintenance and enhancement.

When the functional composition with fatigue accumulation preventing activity is used as a fatigue accumulation preventing composition, it contributes to improvement of voluntary motor activity, relief from fatigue, stress prevention, and maintenance of stamina. It is highly effective to prevent fatigue accumulation compared with foods merely containing soy milk. That is, it produces a novel effect obviously different from the effect obtained through ingestion of proteins or amino acids isolated from soy milk. The functional composition manifests fatigue accumulation preventing activity in the form of an increase in voluntary motor activity which will lead to improvement and maintenance of motor function, increase of muscle mass, improvement of cardiopulmonary function, prevention of obesity, promoted relief from fatigue, nutritional fortification, and prevention of dementia.

When circadian rhythm is disrupted as a result of sleep disorder, insomnia, autonomic dystonia, bipolar disorder, senile dementia, physical disconditioning due to irregular hours (night work, shift work, or long-distance flight), or exhaustion as a result of irregular living habits observed in autism, ingestion of the functional composition having circadian rhythm regulatory activity is effective to normalize the circadian rhythm.

When the functional composition having reproductive behavior inducing activity is used as a reproductive behavior inducing composition, it enhances reproductive behavior to improve sexual function, enhance sexual drive, and improve QOL.

Prepared from soybean, a historically eaten crop, the functional composition of the invention is safe and inexpensive and can be eaten as a daily food having the various activities described.

### BRIEF DESCRIPTION OF THE DRAWING

FIG. 1 is a graph showing the measurements of growth hormone (GH) concentration in GH-secreting cell line culture media in Example 1.

FIG. 2 is a graph showing the measurements of GH concentration in primary pituitary cell culture media in Example 2.

FIG. 3 is a graph showing blood GH level changes in rats following a single administration in Example 3.

FIG. 4 is a graph showing the measurements of blood GH level in rats following continued administration in Example 4.

FIG. 5 is a graph showing the measurements of the number of wheel revolutions by rats given a lactic acid fermented soy milk product or soy milk.

FIG. 6 is a diagram showing the intracellular circadian rhythm controlling mechanism and changes in circadian rhythm controlling gene mRNA expression in rat liver given a lactic acid fermented soy milk product (relative to control) in Example 6.

FIG. 7 is a graph showing the amount of Rev-erb α mRNA expression in rat liver given a lactic acid fermented soy milk product in Example 6.

FIG. 8 is a graph showing the amount of PPAR α mRNA expression in rat liver given a lactic acid fermented soy milk product in Example 7.

FIG. 9 is a graph showing the measurements of blood triglyceride level in rats given a lactic acid fermented soy milk product in Example 8.

FIG. 10 is a graph showing diurnal changes of Rev-erb α mRNA expression in rat liver given soy milk or a lactic acid fermented soy milk product in Example 9.

FIG. 11 is a graph showing diurnal changes of Clock mRNA expression in rat liver given soy milk or a lactic acid fermented soy milk product in Example 9.

FIG. 12 is a graph showing diurnal changes of Bmal 1 mRNA expression in rat liver given soy milk or a lactic acid fermented soy milk product in Example 9.

FIG. 13 is a graph showing the amount of Bmal 1 mRNA expression in rat liver given soy milk or a lactic acid fermented soy milk product at 19:00 h (Zeitgeber time (ZT) 12) in Example 9.

FIG. 14 is a graph showing the number of copulations in rats given distilled water or a lactic acid fermented soy milk product in Example 10.

### BEST MODE FOR CARRYING OUT THE INVENTION

The component of soybean origin (hereinafter simply referred to as a soybean component) that is used to prepare a lactic acid fermented product as an active ingredient of the functional composition of the present invention can be soybean itself or a processed soybean product. Examples of the processed soybean product include soybean meal, soybean flour, soy milk, soybean curd refuse, and soybean extract obtained by extracting soybeans with an appropriate solvent. Soy milk, being a liquid, is particularly preferred because of the ease of handling and suitability to uniform and large-volume culturing. Soy milk is a slurry obtained by grinding water-soaked and swollen soybeans or a liquid after separating solid matter (curd refuse) from the slurry. Various commercially available soy milk products may be used. The cultivar of soybean to be used is not limited and exemplified by Ryuho and Tachiyutaka.

The lactic acid bacteria belonging to the genera Lactobacillus, Leuconostoc, and Pediococcus that can be used in the invention are not particularly limited.

Examples of lactic acid bacteria of the genus Lactobacillus include L. casei, L. plantarum, and L. kefiri, more specifically, L. casei subsp. Casei NRIC 1597, L. plantarum NRIC 1931, and L. kefiri NRIC 1593.

Examples of lactic acid bacteria of the genus Leuconostoc include L. mesenteroides subsp. mesenteroides, L. paramesenteroides, L. pseudomesenteroides, L. citreum, and L. mesenteroides subsp. dextranicum, more specifically, L. pseudomesenteroides ATCC 12291, L. citreum NRIC 1579, L. paramesenteroides NISL 7218, L. mesenteroides subsp. mesenteroides NISL 7201, and L. mesenteroides subsp. dextranicum NRIC 1539.

Examples of lactic acid bacteria of the genus Pediococcus include P. pentosaceus and P. acidilactici, more specifically, P. pentosaceus NRIC 0122 and P. acidilactici NRIC 0124.

Of the above recited lactic acid bacteria particularly preferred is Leuconostoc pseudomesenteroides ATCC 12291.

As used herein, the designation “NRIC” refers to NODAI Research Institute Culture Collection, Faculty of Applied Bio-Science, Tokyo University of Agriculture located at Sakuragaoka 1-1-1, Setagaya-ku, Tokyo, Japan. The designation “NISL” refers to Noda Institute for Scientific Research located at Noda 399, Noda-shi, Chiba, Japan. The designation “ATCC” refers to the American Type Culture Collection depository located at 10801 University Blvd., Manassas, Va. 20110-2209, USA. The strains recited above are available from the indicated collections.

The active ingredient of the functional composition of the invention, a lactic acid fermented product of a soybean component, is obtained by fermenting the above described soybean component by the inoculation of the lactic acid bacterium.

The conditions for lactic acid fermentation are not particularly limited and may be decided as appropriate to the soybean component or lactic acid bacterium used. For example, In using soy milk as a soybean component, a suspension of preculture cells of a lactic acid bacterium is inoculated to soy milk to ferment soy milk under conditions suitable for the lactic acid bacterium, e.g., at 250 to 35° C. for at least 4 hours. In using an anaerobic lactic acid bacterium, fermentation is carried out under an anaerobic condition. The fermentation may be mixed fermentation using two or more lactic acid bacteria or continuous fermentation. Necessary nutrient sources such as at least one sugar selected from sucrose, maltose, stachyose, and raffinose may be added to soy milk to increase fermentation properties.

The resulting fermented product may be used as such as an active ingredient (lactic acid fermented product) of the functional composition of the invention. Optionally, the fermented product as obtained may be fractionated to give a fraction with a higher desired activity for use as an active ingredient of the functional composition. The fractionation is performed by, for example, solvent extraction, gel permeation chromatography or ultrafiltration.

Containing the above described lactic acid fermented product as an active ingredient, the functional composition of the invention exhibits various activities including growth hormone secretagogue activity, fatigue accumulation preventing activity, circadian rhythm regulatory activity, and reproductive behavior inducing activity and is useful as a growth hormone secretagogue, a fatigue accumulation preventing composition, a circadian rhythm regulator, a reproductive behavior inducing composition, and so forth.

The functional composition may be used in the form of a fermented product as produced by the lactic acid fermentation or as formulated into preparations, generally oral preparations.

In formulating into oral preparations, any form and method of formulation can be chosen as appropriate according to the intended use. Examples of suitable preparation forms include tablets, powders, granules, emulsions, pastes, liquids, syrups, and capsules. The functional composition can be formulated into these preparation forms together with pharmaceutically acceptable carriers and vehicles in accordance with customary methods.

The formulations of the functional composition may further contain optional additives or components such as amino acids, fatty acids, crude drugs, saccharides, vitamins, minerals, colorants, flavors, and the like according to the purpose of use.

The functional composition may be processed into the form of a food, either solid or liquid. In this case, the functional composition may be incorporated into any other foods including meat products, marine processed foods, processed vegetables, processed fruits, delicatessen foods, processed soy products, edible flours, edible proteins, beverages, energy drinks, alcoholic drinks, seasonings, dairy products, and confectionery products. The functional composition may be added at any stage of the production of these foods or added to the finished foods.

The fermented product as obtained by the lactic acid fermentation may be supplied as it is as a functional foodstuff with various activities described. The fermented product as obtained may be supplied as a blend with processed vegetables (e.g., tomato, carrot, onion, garlic and sesame) or fruits (e.g., apple, citrus fruits (e.g., Citrus junos and Citrus unshiu Marc.), orange, peach, strawberry, pineapple, and grape) in the form of puree, paste, cut or ground pieces, powder or juice, saccharides, starches, vitamins, minerals, fibers, and so on.

The functional composition can also be used as a food material or an ingredient of foods.

The content of the lactic acid fermented product in the functional composition is not particularly limited and is adjustable according to the purpose and mode of use of the composition, the form of the composition, the kind of the food to which it is incorporated, the age, sex and weight of an ingester, the symptom the composition is intended to treat, and the like.

For instance, when the functional composition is supplied as a growth hormone secretagogue, a fatigue accumulation preventing composition, a reproductive behavior inducing composition or a circadian rhythm regulator, the content of the lactic acid fermented product in the composition is preferably selected according to the age and weight of an ingester, the symptoms to be treated, the number of ingestions, and the like. Taking one to a few ingestions per day by an adult weighing 60 kg, for instance, a recommended content of the lactic acid fermented product in the functional composition would be such that the intake of the fermented product per day may range from about 100 to 1000 g (corresponding to about 1.6 to 16 g/kg BW), preferably 100 to 200 g.

In the cases where the functional composition is provided as incorporated into other foods, the content of the fermented product in the functional composition is preferably decided taking into consideration the amount of the food generally ingested. While depending on the food, the functional composition is preferably added to give a fermented product content of 5 to 50 parts by mass, more preferably 2.5 to 10 parts by mass, per 100 parts by mass of the food.

The functional composition produces markedly excellent effects on continued ingestion. The functional composition is used not only for humans but also as pet foods, animal feeds, or ingredients thereof.

### EXAMPLES

In Examples 1 to 4, soy milk was fermented with a lactic acid bacterium, and the resulting fermented product was tested for growth hormone secretagogue activity.

### Example 1

### (a) Preparation of Lactic Acid Fermented Soy Milk

A soy milk medium consisting of 50% (by mass, hereinafter the same) of soy milk for tofu making (available from Kibun Food Chemifa Co., Ltd.) and 50% of a 40% sucrose aqueous solution (sterilized at 121° C. for 15 minutes) was inoculated with 10⁶ to 10⁷ cfu/ml of each one of the lactic acid bacteria below and incubated at 30° C. for 24 hours to obtain five kinds of fermented soy milk products.

(1) Lactobacillus casei subsp. casei NRIC 1597
(2) Lactobacillus plantarum NRIC 1931
(3) Leuconostoc mesenteroides subsp. mesenteroides NISL 7201
(4) Leuconostoc paramesenteroides NISL 7218
(5) Leuconostoc pseudomesenteroides ATCC 12291

### (b) Assay on Growth Hormone (GH) Secreting Cell Line

MtT/S cells were used as a GH-secreting cell line. To the cell culture was added distilled water (control), soy milk (comparison) or the lactic acid fermented soy milk prepared above in a concentration of 0.1%. Ten minutes later, the culture supernatant was collected, and the GH concentration was measured by ELISA using a rat growth hormone enzyme immunoassay kit (Amersham).

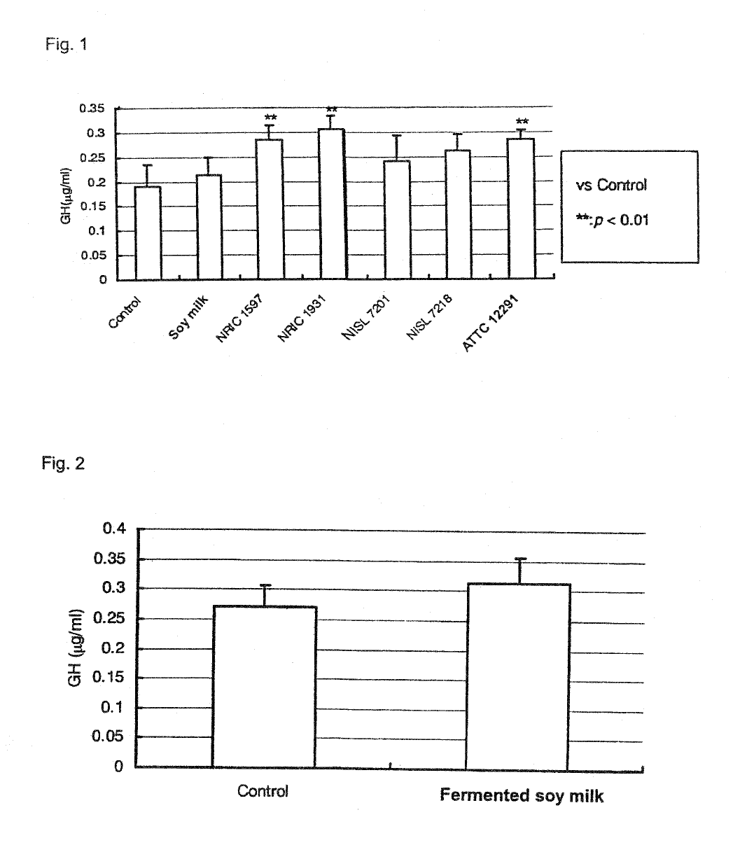

The results obtained are shown in FIG. 1. Addition of the lactic acid fermented soy milk results in an increase of GH concentration in the cell culture supernatant. Compared with addition of soy milk, addition of the lactic acid fermented soy milk tends to result in a higher GH concentration in the cell culture supernatant. The lactic acid fermented soy milk was thus proved to have GH secretagogue activity.

### Example 2

### Assay on Primary Cultured Pituitary Cells

The GH secretagogue activity of the lactic acid fermented soy milk was confirmed by carrying out the assay using primary cultured pituitary cells.

The fermented soy milk obtained by using Leuconostoc pseudomesenteroides ATCC 12291 in Example 1 was used.

The pituitary gland was excised from a Wistar male rat, dissociated with trypsin, and cultured. On the third day of culturing, distilled water (control) or the lactic acid fermented soy milk was added to the cell culture in a concentration of 0.1%. Ten minutes later, the culture supernatant was collected, and the GH concentration was measured by ELISA.

The results obtained are shown in FIG. 2. Addition of the lactic acid fermented soy milk results in an increase of GH concentration in the cell culture medium, proving that the lactic acid fermented soy milk exhibits GH secretagogue activity in the primary cultured pituitary cell system.

### Example 3

### (a) Preparation of Lactic Acid Fermented Soy Milk

A soy milk medium consisting of 80 ml of plain soy milk (available from Kibun Food Chemifa Co., Ltd.) and 5 ml of an 80% glucose aqueous solution (sterilized at 121° C. for 15 minutes) was inoculated with 10⁶ to 10⁷ cfu/ml of Pediococcus pentosaceus NRIC 0122 and incubated at 30° C. for 24 hours to obtain lactic acid fermented soy milk.

### (b) Single Oral Administration in Rat

Ten-week-old Wister male rats, with a cannula placed in the jugular vein for blood sampling, were orally given 10 ml/kg BW of the fermented soymilk or water (control) through a gastric probe. After 30 minutes up to 120 minutes from the administration, blood samples were collected via the cannula every 5 minutes to determine the blood GH levels by ELISA.

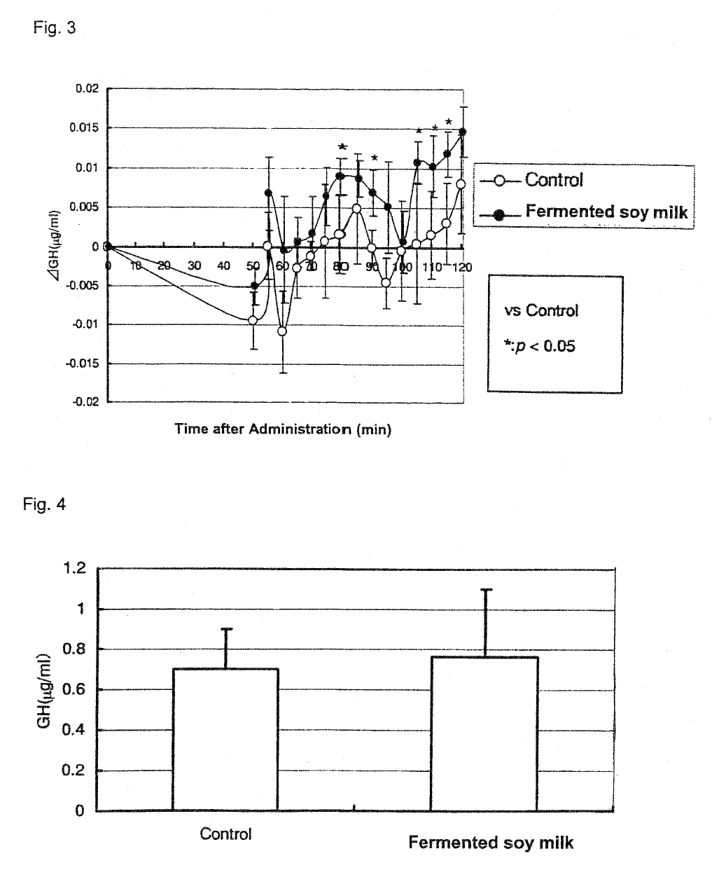

The results obtained are shown in FIG. 3. An increase in blood GH level is observed at 80, 90, 105, 110, and 115 minutes after the administration of the lactic acid fermented soy milk. The lactic acid fermented soy milk was thus proved to have GH secretagogue activity following a single oral administration in rat.

### Example 4

### Continued Administration in Rat

The lactic acid fermented soy milk obtained by using Leuconostoc pseudomesenteroides ATCC 12291 in Example 1 was tested.

Five-week-old Wistar rats were fed ad libitum on a diet containing 5% of the fermented soy milk or a standard diet as a control for 13 weeks. After that, blood was collected from the abdominal aorta under anesthesia, and the blood GH level was measured by ELISA.

The results obtained are shown in FIG. 4. The blood GH level in the rats fed on the fermented soy milk-containing diet is higher than that of the control. The lactic acid fermented soy milk exhibited GH secretagogue activity following continued administrate in rat.

In Example 5, lactic acid fermented soy milk was tested for fatigue accumulation preventing activity.

### Example 5

The lactic acid fermented soy milk obtained by using Leuconostoc pseudomesenteroides ATCC 12291 in Example 1 was used.

Five-week-old Fischer 344 male rats were individually placed in a running wheel cage and fed on a standard diet (MF, from Oriental Yeast Co., Ltd.) ad lib. for 3 weeks, and the number of wheel revolutions per 24 hours was recorded. The rats were then fed ad lib. on MF containing 5% of the lactic acid fermented soy milk (fermented soy milk-containing diet) or, for comparison, MF containing 5% of soy milk (soy milk-containing diet) for an additional 3 week period, and the number of wheel revolutions was recorded in the same manner. Thereafter, the rats were fed on MF ad lib. for 3 more weeks, during which the number of wheel revolutions was recorded.

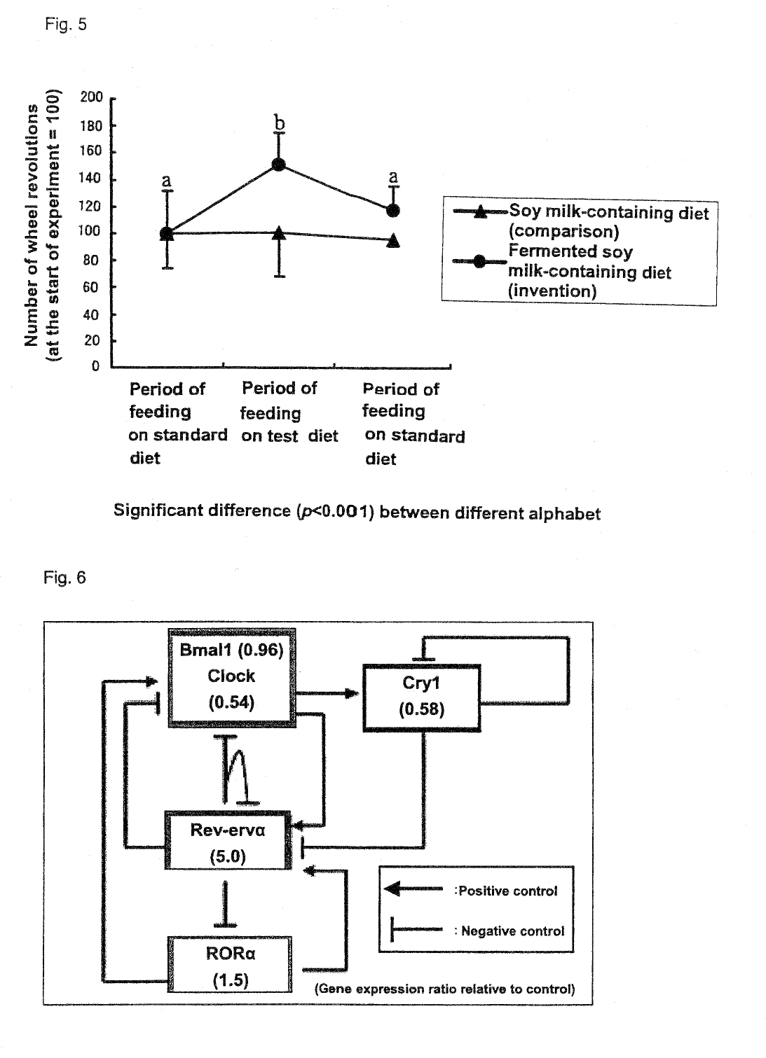

The results obtained are shown in FIG. 5. Changing from the standard diet to the fermented soy milk-containing diet results in a significant increase in the number of revolutions of the running wheel. The change from the fermented soy milk-containing diet to the standard diet results in a significant decrease in the number of revolutions of the running wheel. On the other hand, changing from the standard diet to the soy milk-containing diet (comparison) brings about no difference in the number of wheel revolutions. As can be seen from the graph of FIG. 5, the fermented soy milk-containing diet group shows an increase as great as about 50% in voluntary motor activity over the soy milk-containing diet group, indicating that the lactic acid fermented soy milk largely contributes to prevention of fatigue accumulation. It is thus confirmed that the effect of preventing fatigue accumulation does not owe to the proteins or amino acids of soy milk origin but the lactic acid fermentation product of soy milk.

In Examples 6 to 9, the lactic acid fermented soy milk was tested for circadian rhythm regulatory activity; the circadian rhythm controlling mechanism by the lactic acid fermented soy milk was studied; and diurnal changes in circadian rhythm controlling gene expression caused by the fermented soy milk were compared with those by soy milk.

### Example 6

### DNA Microarray Analysis and Quantitative RT-PCR Analysis

The fermented soy milk obtained by using Leuconostoc pseudomesenteroides ATCC 12291 in Example 1 was used.

Five-week-old Wistar male rats were housed individually (n=5 per group) and given free access to a standard diet (MF) in a control group and to the standard diet to which 5% of the fermented soy milk had been added in a test group for 13 weeks.

The daily food intake per rat was about 20 g. Accordingly, the daily fermented soy milk intake per rat in the test group was 1 g, which is equivalent to 100 to 200 g per day in humans.

After the 13 week feeding, the rats were killed, and the liver was excised immediately. RNA was extracted from the liver, and gene expressions were comprehensively analyzed using a DNA microarray (Rat Genome 230 2.0 Array, from Affymetrix). As for Rev-erb α gene that showed the greatest change of gene expression in the DNA microarray analysis, the RNA sample was subjected to quantitative RT-PCR using glyceraldehyde 3-phosphate dehydrogenase (GAPDH) as an internal control.

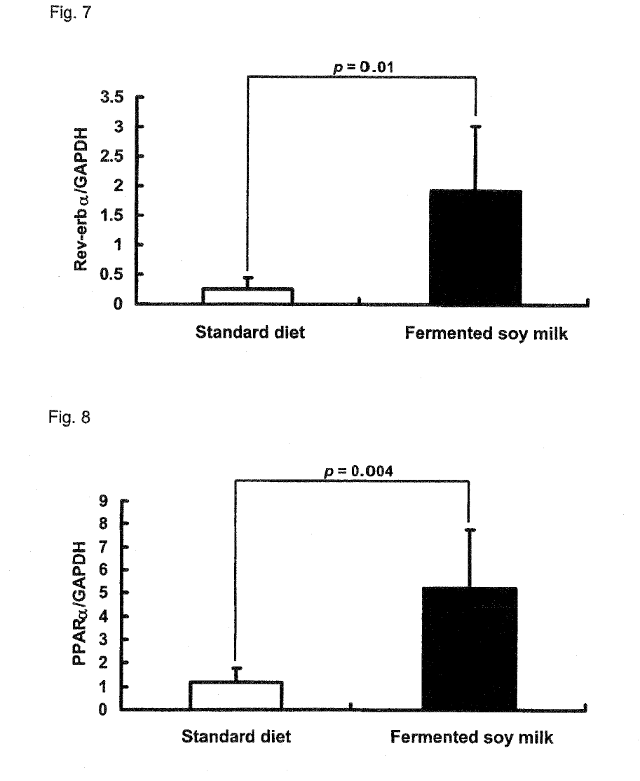

The results obtained are shown in FIGS. 6 and 7. Administration of the functional composition of the invention (the lactic acid fermented soy milk) results in changes of expression of the intracellular circadian rhythm controlling genes in the liver compared with the control (FIG. 6). The results of quantitative RT-PCR assay verifies a significant increase of the Rev-erb α mRNA expression in the liver (FIG. 7), providing a support for the results of the DNA microarray analysis.

### Example 7

### Measurement of PPAR α mRNA Level

The amount of expression of the liver PPAR u gene obtained in Example 6 was measured by quantitative RT-PCR analysis The results are shown in FIG. 8. The PPAR a mRNA expression increases significantly by the administration of the functional composition (the lactic acid fermented soy milk) compared with the control.

### Example 8

### Measurement of Blood Triglyceride Level

Blood was collected from the rats after the 13 week feeding period in Example 6 and measured for blood triglyceride level using a triglyceride assay kit (Triglyceride G-Test Wako, from Wako Pure Chemical Industries, Ltd.).

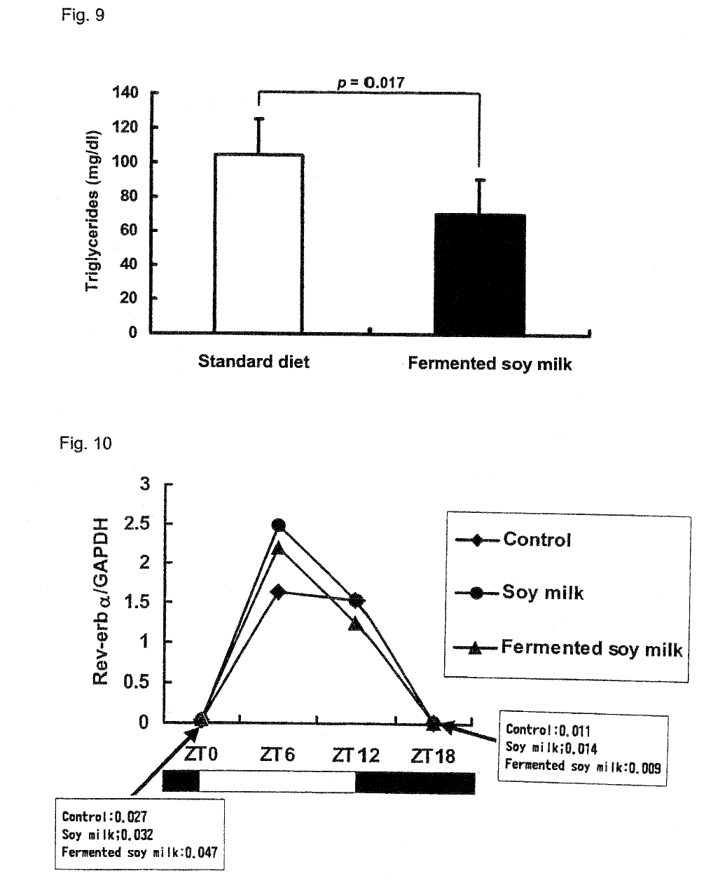

The results obtained are shown in FIG. 9. The blood triglyceride level decreases significantly by the administration of the functional composition (the lactic acid fermented soy milk) compared with the control.

### Example 9

### Study on Diurnal Changes of Circadian Rhythm Controlling Gene Expression

The fermented soy milk obtained by using Leuconostoc pseudomesenteroides ATCC 12291 in Example 1 was used.

Nine-week-old Wistar male rats were housed individually (n=20 per group) in a conventional room (light time: 7:00-19:00; dark time: 19:00-7:00). The rats were fed ad libitum on a standard diet (MF) in a control group, the standard diet to which 5% of soy milk had been added in a soy milk group, and the standard diet to which 5% of the fermented soy milk had been added in a fermented soy milk group all for 4 weeks. After the 4-week administration period, five animals per group were killed, and the liver was excised immediately at 7:00 (ZT0), 13:00 (ZT6), 19:00 (ZT12) and 1:00 (ZT18). RNA was extracted from the liver and subjected to quantitative RT-PCR using primers specific for the circadian rhythm controlling genes. GAPDH was used as an internal control as in Example 6.

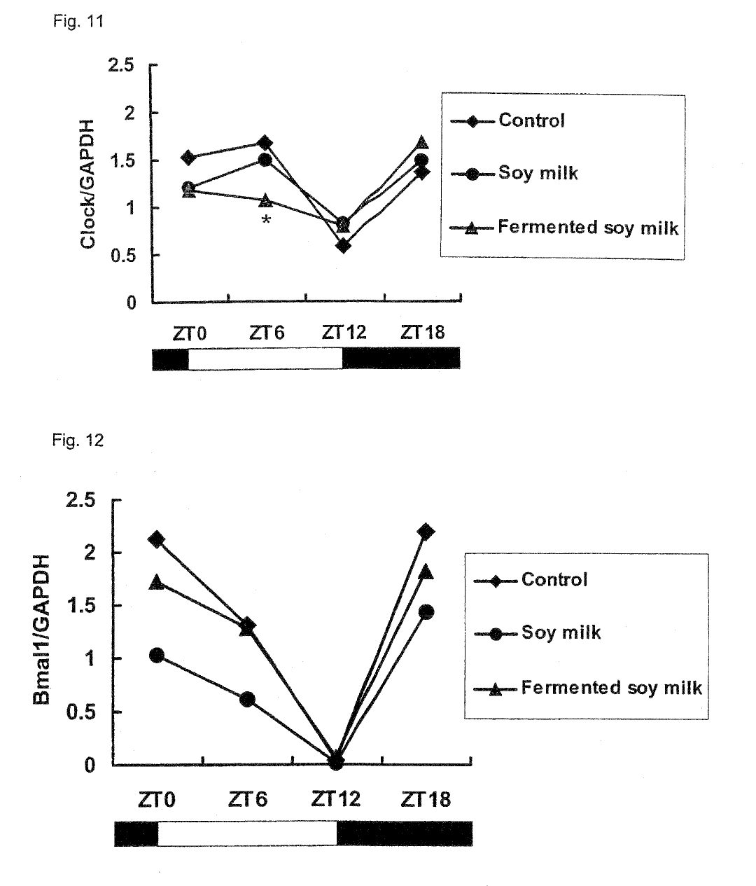

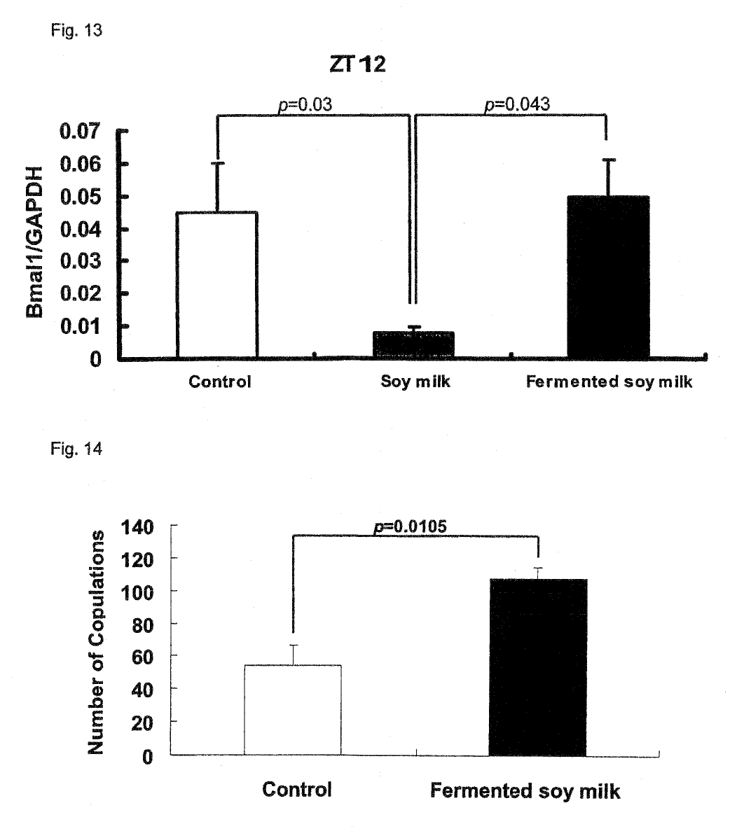

The results obtained are shown in FIGS. 10 through 13. As compared with the control group, the soy milk group and the fermented soy milk group tend to show changes in the Rev-erb a mRNA expression profile (FIG. 10). The fermented soy milk group shows a significant reduction in Clock mRNA expression at ZT6 (FIG. 11). On the other hand, the soy milk group tends to have lower Bmal 1 mRNA expression whole day and shows a lower expression than the control and fermented soy milk groups at ZT12 (FIGS. 12 and 13). All these results provide confirmation that soy milk and the lactic acid fermented soy milk have different influences on the circadian rhythm controlling gene expression profiles in rat.

### Example 10

In this example, the reproductive behavior inducing activity of lactic acid fermented soy milk was tested.

### (a) Preparation of Lactic Acid Fermented Soy Milk

A soy milk medium consisting of 50% of soy milk for tofu making (from Kibun Food Chemifa Co., Ltd.) and 50% of a 40% sucrose aqueous solution (sterilized at 121° C. for 15 minutes) was inoculated with 10⁶ to 10⁷ cfu/ml of Leuconostoc pseudomesenteroides ATCC 12291 and incubated at 30° C. for 24 hours to obtain fermented soy milk.

### (b) Measurement of the Number of Copulations

Nine-week-old Wistar male rats were housed in individual wire cages with a 12-hour light/dark cycle (light time: 7:00-19:00, dark time: 19:00-7:00). The rats were divided into a control group (n=5) orally administered with distilled water and a group (n=5) orally administered with 3.3 g/kg BW/day of the fermented soy milk. The oral administration was carried out by means of a gastric probe between 8:00 and 10:00 AM. Each one of the male rats and four female rats of the same age as the male rats were housed in a polycarbonate cage between 19:00 to 7:00 on the tenth day from the start of administration. The behavior of the rats was videotaped using red light (5W), and the number of copulations during the 12 hour period was counted.

The results are shown in FIG. 14. The number of copulations of the male rats in the fermented soy milk group significantly increases over that of the control group, proving that the lactic acid fermented product of soy milk (a soybean component) exhibits reproductive behavior inducing activity.

### INDUSTRIAL APPLICABILITY

The functional composition of the present invention has various activities including growth hormone secretagogue activity, fatigue accumulation preventing activity, circadian rhythm regulatory activity, and reproductive behavior inducing activity and is highly safe, inexpensive, and easy to ingest. It is useful as a food, a food material, or a pharmaceutical.

The functional composition can be used, for example, as added to any food, either solid or liquid, to easily provide special health foods or functional foods exhibiting physiological activities such as increase in muscle mass, skin elasticity, and bone density through the growth hormone secretagogue action; special health foods or functional foods manifesting the fatigue accumulation preventing effects in the form of promotion of voluntary motor activity, improvement and maintenance of motor function, increase of muscle mass, improvement of cardiopulmonary function, prevention of obesity, provision of relief from fatigue, nutritional fortification, and prevention of dementia; special health foods or functional foods effective in treating and preventing sleep disorder, insomnia, and bipolar disorder through the circadian rhythm regulatory activity; and special health foods or functional foods showing the reproductive behavior inducing effects in the form of improvement on reproductive function, sexual drive enhancement, and improvement on QOL.

1. A functional composition containing as an active ingredient a lactic acid fermented product obtained by fermenting a component of soybean origin using a lactic acid bacterium belonging to the genera Lactobacillus, Leuconostoc or Pediococcus.

2. The functional composition according to claim 1, wherein the component of soybean origin is soy milk.

3. The functional composition according to claim 1, wherein the lactic acid bacterium is Leuconostoc pseudomesenteroides ATCC 12291.

4. The functional composition according to claim 1, which is a functional food.

5. The functional composition according to claim 1, which is a growth hormone secretagogue.

6. The functional composition according to claim 1, which is a fatigue accumulation preventing composition.

7. The functional composition according to claim 1, which is a reproductive behavior inducing composition.

8. The functional composition according to claim 1, which is a composition regulating circadian rhythm through an increase of Rev-erb α and/or PPAR α mRNA expression or a decrease of Clock or Bmal 1 mRNA expression.

9. The functional composition according to claim 8, which is used to prevent or reduce a symptom attributed to disrupted circadian rhythm.

10. The functional composition according to claim 9, wherein the symptom attributed to disrupted circadian rhythm is sleep disorder, insomnia, autonomic dystonia, bipolar disorder, or jet lag.

11. A process of preparing the functional composition according to claim 1, comprising inoculating a lactic acid bacterium belonging to the genera Lactobacillus, Leuconostoc, or Pediococcus to a component of soybean origin, then subjecting the component of soybean origin to fermentation, and collecting the fermented product.

12. The process according to claim 11, wherein the lactic acid bacterium is Leuconostoc pseudomesenteroides ATCC 12291.

13. The functional composition according to claim 2, wherein the lactic acid bacterium is Leuconostoc pseudomesenteroides ATCC 12291.
